(19) United States
(12) Patent Application Publication
(10) Pub. No.: US 2026/0363277 A1
(43) Pub. Date: Nov. 19, 2026

(54) High Fiber, Low Sugar Food And Beverage Products

(71) Applicant: Amanda Freeman, North Providence, RI (US)

(72) Inventor: Amanda Freeman, North Providence, RI (US)

(21) Appl. No.: 19/679,671

(22) Filed: May 16, 2026

Related U.S. Application Data
(60) Provisional application No. 63/806,948, filed on May 16, 2025.

(51) Int. Cl. A23L 2/385; A23L 2/08; A23L 2/56; A23L 2/60; A61K 9/00; A61K 31/736; A61K 36/48; A61K 47/12; A61K 47/46

(52) U.S. Cl. CPC A23L 2/385; A23L 2/08; A23L 2/56; A23L 2/60; A61K 9/0095; A61K 31/736; A61K 36/48; A61K 47/12; A61K 47/46

(57) ABSTRACT

High fiber and low sugar food and beverage products are provided. The high fiber and low sugar food and beverage products are particularly suitable for children. The food and beverage products of the invention have many health benefits including, but not limited to, supporting digestive health, helping to manage weight gain, and regulating blood sugar.

### CROSS-REFERENCE TO RELATED APPLICATIONS

The application claims the benefit of U.S. Provisional Patent Application No. 63/806,948 filed May 16, 2025, the entire disclosure of which is hereby incorporated by reference.

### BACKGROUND OF THE INVENTION

### Field of the Invention

The present invention generally relates to food and beverage products. More particularly, the invention relates to high fiber and low sugar food and beverage products, especially suitable for children. The beverage products of the invention have many health benefits including, but not limited to, supporting digestive health, helping to manage weight gain, and regulating blood sugar levels.

### Brief Review of the Related Art

In recent years, advanced medical and nutritional research has identified more childhood health issues relating to low fiber diets. A lack of fiber in the diet has been associated with several disorders in children including constipation, irritable bowel syndrome, allergies and immune-related disorders, obesity, and type 2 diabetes. In general, there are two different types of fiber: 1) soluble fiber which retains water and is passed slowly through the digestive tract; and 2) insoluble fiber which does not retain water and passes through the digestive tract largely intact.

Soluble fiber generally follows a slow passage through the digestive system and allows more time for the food to be absorbed. Soluble fiber can help prevent excess weight gain, lower cholesterol, and maintain steady blood sugar. Insoluble fiber helps with constipation and helps food pass quickly through the stomach and intestines.

High fiber diets have become more popular, and many high-fiber food products have been developed. For example, Schmidt, U.S. Pat. No. 4,711,786 discloses high fiber bakery products such as bread, buns, rolls, and croissants. According to the '786 patent, the bakery products are higher in fiber than whole wheat bread and higher in protein than normally found in bakery items. The composition comprises flour, water, sugar, shortening, yeast and salt together with two or more of isolated pea protein, pea flour (or ground pea cotyledons), ground triticale hulls and ground pea hulls. A premix consisting of pea fiber and triticale fiber may be mixed with wheat flour and other ingredients to make high fiber bakery items or with wheat flour or various other types of cereal grain flours to make an extruded edible product such as a pasta, a snack product or a cereal.

Hodgson et al., U.S. Pat. No. 4,824,683 discloses a low calorie, high fiber bread product. According to the '683 patent, the bread products have the aroma and tender eating characteristics of bread as well as a reduction of available calories compared with conventional bread. The composition for use in the making of the high fiber content bread contains the usual baker's flour and about 10 to 40 parts by weight based on flour (baker's flour) of pre-gelatinized pea bran and 10 to 40 parts by weight based on flour of pre-gelatinized oat bran

Schmidt, U.S. Pat. No. 5,275,833 discloses a process for bleaching edible green fibrous vegetable products. The fibrous material is immersed in an aqueous dispersion of chlorine for a period of time with a concentration of chlorine that is effective for removing substantially all of the coloration from the material with a minimal effect on the fiber matrix,

Levin et all., U.S. Pat. No. 7,252,850 discloses additives for increasing the fiber content of bread products are provided. The fiber additives comprise protein, hydrocolloids, and oil and optionally may contain minerals and emulsifiers. Preferred additives according to the '850 patent comprise dietary fiber, guar gum, xanthan gum, calcium carbonate, lecithin and canola oil. These additives are processed according to the '850 patent to form a high-density complex composition having a dietary fiber content of about 85% by weight.

Although some high fiber food and beverage products have some health benefits, there is a need for new fiber-rich foods and beverages that are particularly helpful for children's growth and development. There is a particular need for high fiber beverage products having desirable flavoring that children will want to consume.

The present invention provides such food and beverage products. The food and beverage products of the invention have high fiber and low sugar concentrations. The food and beverages have many health benefits including, but not limited to, supporting digestive health, helping to manage weight gain, and regulating blood sugar levels. In addition, because fiber-rich foods help to reduce cholesterol levels, these foods and beverages may help with improving heart health. Also, the food and beverage products of the invention help strengthen immunity by providing a more balanced and healthy gut biome. Other features, benefits, and advantages of the present invention are described further below.

### SUMMARY OF THE INVENTION

The present invention generally relates to high fiber food and beverage products, particularly for children. The food and beverage products of the invention have many health benefits including, but not limited to, supporting digestive health, helping to manage weight gain, and regulating blood sugar.

In one embodiment, the beverage composition comprises pear juice concentrate, grape juice concentrate, lemon juice concentrate, and acacia powder and partially hydrolyzed guar gum, wherein the ratio of acacia powder to partially hydrolyzed guar gum is 1:1. Preferably, the ratio of pear juice concentrate to grape juice concentrate is also 1:1, and the ratio of acacia powder to partially hydrolyzed guar gum is 1:1. Preferably, the acacia powder is organic. The composition can also comprise flavoring agents and sweeteners selected from the group consisting of lemon juice concentrate, monksweet, tropical cooler, strawberry extract, and fruit punch, and combinations thereof.

### DETAILED DESCRIPTION OF THE INVENTION

In one embodiment of the present invention, a fiber-rich beverage is provided. Preferably, the fiber-rich composition has a minimal or zero amount of sugar. The present invention is illustrated by the following example, but this example should not be construed as limiting the scope of the invention.

TABLE 1
Beverage Formulation

| | Amount | | Weight |
|---|---|---|---|
| Ingredient | (Grams) | | Percentage (%) |
| Pure Filtered Water | 800-1200 | gms. | 89.00-94.00% |
| Pear Juice Concentrate* | 17-22 | gms. | 1.00-2.50% |
| Grape Juice Concentrate* | 17-22 | gms. | 1.00-2.50% |
| Lemon Juice Concentrate** | 18-23 | gms | 1.00-3.00% |
| Monksweet LS*** | 0.10-2 | gms. | 0.01-1.00% |
| Cane Sugar | 1-10 | gms. | 0.1-2.00% |
| Acacia Powder **** | 13-21 | gms. | 1.00-2.00% |
| PHGG ***** | 13-21 | gms. | 1.00-2.00% |
| Tropical Cooler flavoring agent | 0.1-2 | gms. | 0.01-1.50% |
| Strawberry Extract | 0.1-1.5 | gms. | 0.01-1.00% |
| Citric Acid | 0.1-1.5 | gms. | 0.01-1.00% |
| Fruit Punch flavoring agent | 0.1-2 | gms. | 0.01-1.50% |

*Pear Juice and Grape Juice Concentrates - Preferably, aseptic pear and grape juices are used. These shelf-stable products are processed using aseptic packaging, ensuring they remain sterile without refrigeration until they are opened. These juices have low acidity and help regulate digestion. The concentrates are natural sweeteners and flavoring agents produced from evaporating water from the juices. Available from Nature's Flavors (Anaheim, CA).
**Lemon Juice Concentrate - Flavoring and sweetening agent having a high content of Vitamin C. Available from Icon Foods (Salem, OR).
***Monksweet LS - Natural, zero-calorie powdered sweetener blend containing monk fruit extract (Mongroside V) and Stevia ™ (Rebaudioside A). Available from Icon Foods (Portland, OR).
**** Acacia Powder - Prebiotic soluble fiber derived from the sap of the Acacia Senegal tree that supports digestive health and promotes a healthy gut microbiome. Preferably, Organic Acacia Powder is used.
***** PHGG - Partially Hydrolyzed Guar Gum that acts as a soluble, prebiotic dietary fiber for improving gut health, digestive regularity, and glycemic control.

All of the ingredients for the beverage formulation provided in above Table 1 were mixed in a blender and heated to 185 °F to prepare the final beverage. The resulting beverage provides many health benefits including, but not limited to: a) reducing constipation—bowel movements tend to be more consistent and softer; b) helping to reduce bloating by supporting a more healthy microbiome—a more beneficial and balanced bacteria community is provided in the gut; c) boosting the microbiome in the gut—this helps with digestion and building-up immunity, and even mental health; d) stabilizing blood sugar levels; and e) helping to reduce inflammation so there can be healthier skin. There may also be improvements in energy and mental clarity

The beverage formulations of the present invention can have a wide variety of flavors. Children particularly enjoy flavored beverages. For example, a Lemon Berry Splash flavored beverage can be made with natural lemon and raspberry extracts, and a touch of monk fruit with a pink hue that is provided from a natural fruit or vegetable color (such as elderberry or beet).

A Peach Mango Chill flavored beverage can be made having a natural peach and mango flavor with a light stevia blend.

A Grape Pop flavored beverage can be made. The beverage can include concord grape essence with a slight fizz or it can be still. A natural color can be provided with a natural fruit or vegetable color (such as purple carrot or blueberry).

An Orange Cream flavored beverage can be made. The beverage can include natural orange oil and vanilla.

A Cucumber Lime flavored beverage can be made. The beverage can include crisp cucumber and lime oil.

A Watermelon Mint flavored beverage can be made. The beverage can include natural watermelon and a hint of mint.

In addition, the present invention includes the following fiber-rich food compositions that are particularly suitable for children.

In one preferred embodiment, the beverage product described above is particularly effective because the Organic Acacia powder effective and Partially Hydrolyzed Guar Gum (PHGG) powder completely dissolve into the beverage liquid. That is, there is no gelling of the powders. The completely dissolved powder can be easily mixed into the beverage to provide a high fiber, low sugar beverage that supports digestive health and promotes a healthy gut microbiome.

It should be understood that the food and beverage products, compositions, formulations, materials, and the like described and illustrated herein represent only some embodiments of the invention. It is appreciated by those skilled in the art that various changes and additions can be made to the food and beverage products, compositions, formulations, materials, and the like herein without departing from the spirit and scope of this invention.

I claim:

1. A composition for making a beverage having a high fiber content beverage, the composition comprising pear juice concentrate, grape juice concentrate, lemon juice concentrate, and acacia powder and partially hydrolyzed guar gum, wherein the ratio of acacia powder to partially hydrolyzed guar gum is 1:1.

2. The composition of claim 1, wherein the ratio of pear juice concentrate to grape juice concentrate is 1:1.

3. The composition of claim 1, wherein the ratio of acacia powder to partially hydrolyzed guar gum is 1:1.

4. The composition of claim 1, wherein the acacia powder is organic.

5. The composition of claim 1, wherein the composition further comprises flavoring agents and sweeteners selected from the group consisting of lemon juice concentrate, monksweet, tropical cooler, strawberry extract, and fruit punch, and combinations thereof.
